(19) United States
(12) Patent Application Publication
(10) Pub. No.: US 2017/0128468 A1
(43) Pub. Date: May 11, 2017

(54) PHOSPHATIDYLGLYCEROL AS AN ANTI-INFLAMMATORY

(71) Applicant: Agusta University Research Institute, Inc., Augusta, GA (US)

(72) Inventors: Wendy Bollag, Martinez, GA (US); Vivek Choudhary, Augusta, GA (US); Lawrence Olala, Augusta, GA (US)

(21) Appl. No.: 15/347,491

(22) Filed: Nov. 9, 2016

Related U.S. Application Data
(60) Provisional application No. 62/253,430, filed on Nov. 10, 2015.

(51) Int. Cl. A61K 31/661

(52) U.S. Cl. CPC A61K 31/661

(57) ABSTRACT

Compositions and methods for reducing, or inhibiting inflammation in a subject in need thereof have been developed. Pharmaceutical compositions including one or more phosphatidylglycerol (PG), or functional derivatives thereof, in an effective amount to reduce or inhibit inflammation are provided. The compositions are particularly suited for treating inflammation associated with the skin such as psoriasis, or inflammation associated with the eye. Methods for treating, or preventing inflammation in an inflammatory condition, or an autoimmune disease using the composition, optionally in combination with one or more therapeutic, prophylactic or diagnostic agents, or procedures, are described.

### CROSS-REFERENCE TO RELATED APPLICATIONS

This application claims benefit of U.S. Provisional Application No. 62/253,430 filed Nov. 10, 2015, which is incorporated by reference in its entirety.

### STATEMENT REGARDING FEDERALLY SPONSORED RESEARCH OR DEVELOPMENT

This invention was made with government support under a Small Business Technology Transfer (STTR) Grant No. R41-AR055022 awarded by the National Institutes of Health. The government has certain rights in the invention.

### FIELD OF THE INVENTION

The invention generally relates to compositions and methods for treating, alleviating, or preventing inflammation in an inflammatory disease and/or an autoimmune disorder such as psoriasis.

### BACKGROUND OF THE INVENTION

The skin is the largest organ of the body and is composed of the epidermis and dermis. The most important function of the skin is to provide the essential physical and water permeability barrier. The epidermis is a continuously regenerating tissue, which differentiates to produce a mechanical and water permeability barrier. This barrier is established in the epidermis by a precisely regulated keratinocyte differentiation program that results in distinct epidermal layers. The structure of the epidermis is maintained by a finely tuned balance between keratinocyte proliferation and differentiation, which results in a multilayer structure consisting of basal, spinous, granular, and cornified layers.

The precise regulation of differentiation in the epidermis is crucial for proper stratification and barrier formation to occur. Epidermal homeostasis is maintained in part by orchestrating the correct expression of genes in keratinocytes at each stage of differentiation. Alterations in this differentiation program can result in skin disorders, such as psoriasis. Psoriasis is a polygenic, inherited disease of uncontrolled cutaneous inflammation. Psoriatic skin is characterized by dense infiltrates of T cells and cells of the innate immune system, including neutrophils, dendritic cells, macrophages, and NKT cells. The epithelial cells of the skin are hyperproliferative and fail to undergo normal differentiation, leading to a marked thickening of the epidermis. There are dramatic increases in the number and size of blood vessels situated just below the epidermis, and abscesses composed of neutrophils form within the epidermis. The clinical result is red, thickened, and flaking skin (Clark R A et al., J Clin Invest. 116(8): 2084-2087 (2006)).

The recruitment and activation of macrophages in psoriatic skin appears to be a key pathogenic event in the development and maintenance of psoriatic skin disease. In one mouse psoriasis model, macrophage recruitment and activation were mediated by signals provided by keratinocytes with impaired NF-κB signaling (Stratis A et al., J. Clin. Invest. 116:2094-2104 (2006)). In addition, inflammatory cytokines such as TNF-α produced by these infiltrated macrophages and other cells then drive the development of the skin changes observe in psoriasis (Wang H et al., J. Clin. Invest. 116:2105-2114 (2006)).

Despite many recent advances in understanding inflammatory conditions such as psoriasis, an effective treatment for these chronic inflammatory diseases is still lacking.

Therefore, it is an object of the invention to provide new and effective therapeutic agents and methods for treating inflammatory diseases and disorders.

It is also an objective of the invention to provide ways to control inflammation associated with chronic skin inflammation such as in psoriasis.

It is a further objective to provide new methods and treatments to reduce, or inhibit inflammation caused by cells associated with pathogenesis of psoriasis including keratinocyte and macrophages.

### SUMMARY OF THE INVENTION

Pharmaceutical compositions including an effective amount of one or more phosphatidylglycerol (PG), and methods of use thereof for treating, inhibiting, or preventing inflammation are disclosed. Typically, administration of the compositions is effective to reduce or inhibit inflammation in a subject in need thereof.

In some embodiments, the compositions and methods of use thereof are effective to inhibit, or reduce inflammation caused directly, or indirectly by one or more factors of the Damage Associated Molecular Patterns (DAMPs). In some embodiments, the inflammatory conditions are caused by one or more S100A proteins. In one embodiment, the inflammatory conditions are caused by S100A9.

In some embodiments, the compositions and methods of use thereof are effective to inhibit, or reduce inflammation caused directly, or indirectly by activation through one or more receptors for damage associated molecular patterns (DAMPs). In some embodiments, the composition is effective to inhibit or reduce activation through toll-like receptor (TLR) family members such as TLR-1, TLR-2, TLR-4, TLR-6, or combinations thereof.

In some embodiments, the composition is effective to inhibit, or reduce inflammation caused directly, or indirectly by one or more pro-inflammatory cytokines. Exemplary pro-inflammatory cytokines include IL1α, IL1β, IL6, and TNFα. In some embodiments, compositions are administered to a subject in an effective amount to reduce the expression levels of one or more pro-inflammatory cytokines, reduce the activities of one or more pro-inflammatory cytokines, reduce the secretion of one or more pro-inflammatory cytokines, reduce the ratio of pro-inflammatory cytokines to anti-inflammatory cytokines, or a combination thereof.

In some embodiments, the composition is effective to inhibit, or reduce inflammation caused directly, or indirectly by one or more protein transcription factors. In some embodiments, the compositions are administered to a subject in an effective amount to reduce, or inhibit the activity of one or more protein transcription factors involved in the pathogenesis of the inflammatory conditions such as psoriasis. In one embodiment, the protein transcription factor is NF-κB.

The disclosed compositions and methods are particularly effective for treating skin inflammatory conditions such as psoriasis. Compositions include one or more phosphatidylglycerol molecules in an effective amount to reduce, or inhibit inflammation associated with the psoriasis. In some embodiments, the one or more phosphatidylglycerol molecules are soy phosphatidylglycerol molecules. In some embodiments, the pharmaceutical compositions further include a barrier-disrupting agent, or a penetration-enhancing vehicle for enhanced delivery of the active agents to the site of action.

Compositions and methods of use thereof for reducing or inhibiting inflammation of the mucosal surface such as the eye of a subject include one or more phosphatidylglycerol molecules in an effective amount to inhibit or reduce inflammatory response of the mucosal surface are described.

Methods of treating subjects in need there using these compositions are also provided. Typically, the disclosed compositions are administered topically to a site of inflammation on the subject, preferably a human subject. In some embodiments, the compositions are administered prior to, in conjunction with, subsequent to, or alternation with additional therapy/procedure, for example, treatment with one or more therapeutic, prophylactic or diagnostic agent, and/or light therapy.

In one embodiment, the composition consists of or essentially of phosphatidylglycerol.

### BRIEF DESCRIPTION OF THE DRAWINGS

FIGS. 1A-1D are bar graphs showing mRNA expression levels of inflammatory mediators in keratinocytes after 2-hour incubation in 0 μg/mL Pam₃CSK₄ (Control), 1 μg/mL Pam₃CSK₄ (Pam 1), 2.5 μg/mL Pam₃CSK₄ (Pam 2.5), in the presence or absence of 50 μg/mL dioleoylphosphatidylglycerol (DOPG 50) or 100 μg/mL DOPG (DOPG 100), including (FIG. 1A) IL1α, (FIG. 1B) IL1β, (FIG. 1C) IL6, and (FIG. 1D) TNFα, monitored by quantitative RT-PCR with GAPDH used as the housekeeping gene. Results represent the means±SEM of 4 separate experiments performed in duplicate; *p<0.05, **p<0.01 and ***p<0.001 versus the zero concentration control; ^{fff}p<0.001, τττp<0.001, ^{f}p<0.05, ^{ff}p<0.01 versus the indicated groups.

FIGS. 2A-2D are bar graphs showing mRNA expression levels of inflammatory mediators in keratinocytes after 2-hour incubation in 0 μg/mL Pam₃CSK₄ (Control), 2.5 μg/mL Pam₃CSK₄ (Pam), in the presence or absence of 10 μg/mL soy phosphatidylglycerol (s-PG 10) or 50 μg/mL DOPG (s-PG 50), including (FIG. 2A) IL1α, (FIG. 2B) IL1β, (FIG. 2C) IL6, and (FIG. 2D) TNFα, monitored by quantitative RT-PCR with GAPDH used as the housekeeping gene. Results represent the means±SEM of 4 separate experiments performed in duplicate; *p<0.05, **p<0.01 and ***p<0.001 versus the zero concentration control; ^{fff}p<0.001 versus the indicated groups.

FIGS. 3A-3D are bar graphs showing mRNA expression levels of inflammatory mediators in macrophage cell line RAW 264.7 after 2-hour incubation in 0 μg/mL Pam₃CSK₄ (Control), 1 μg/mL Pam₃CSK₄ (Pam 1), 2.5 μg/mL Pam₃CSK₄ (Pam 2.5), in the presence or absence of 50 μg/mL DOPG (DOPG 50) or 100 μg/mL DOPG (DOPG 100), including (FIG. 3A) IL1α, (FIG. 3B) IL1β, (FIG. 3C) IL6, and (FIG. 3D) TNFα, monitored by quantitative RT-PCR with GAPDH used as the housekeeping gene. Results represent the means±SEM of 4 separate experiments; *p<0.05, **p<0.01 and ***p<0.001 versus the zero concentration control; ^{f}p<0.05, ^{ff}p<0.01, τp<0.05, ξ p<0.05 versus the indicated groups.

FIGS. 4A-4F are bar graphs showing mRNA expression levels of proteins in keratinocytes after 2-hour incubation in 0 μg/mL recombinant S100A9 or lipopolysaccharide (Control), 1 μg/mL recombinant S100A9 (S100A9 1 μg), and 3 μg/mL recombinant S100A9 (S100A9 3 μg), 1 unit of lipopolysaccharide (LPS 1U), or 1000 units of LPS (LPS 1000U), including (FIG. 4A) IL1α, (FIG. 4B) IL1β, (FIG. 4C) IL6, (FIG. 4D) TNFα, (FIG. 4E) TLR1, and (FIG. 4F) TLR2, monitored by quantitative RT-PCR with GAPDH used as the housekeeping gene. Results represent the means±SEM of 3 separate experiments; *p<0.05, **p<0.01 and ***p<0.001 versus the control.

FIGS. 5A-5C are bar graphs showing mRNA expression levels of inflammatory mediators in keratinocytes after 2-hour incubation in control, 3 μg/mL recombinant S100A9 (S100A9), 100 μg/mL DOPG (DOPG), or combination of 3 μg/mL recombinant S100A9 and 100 μg/mL DOPG (S100A9+ DOPG), including (FIG. 5A) IL1β, (FIG. 5B) IL6, and (FIG. 5C) TNFα, monitored by quantitative RT-PCR with GAPDH used as the housekeeping gene. Results represent the means±SEM of 3 separate experiments; **p<0.01 and ***p<0.001 versus all other conditions.

FIGS. 6A-6D are bar graphs showing mRNA expression levels of inflammatory mediators in macrophage cell line RAW 264.7 after 2-hour incubation in control, 2 μg/mL recombinant S100A9 (S100A9), 100 μg/mL DOPG (DOPG), or combination of 2 μg/mL recombinant S100A9 and 100 μg/mL DOPG (S100A9+DOPG), including (FIG. 6A) IL1α, (FIG. 6B) IL1β, (FIG. 6C) IL6, and (FIG. 6D) TNFα, monitored by quantitative RT-PCR with GAPDH used as the housekeeping gene. Results represent the means±SEM of 3 separate experiments; ***p<0.001 versus all other conditions.

FIGS. 7A-7D are bar graphs showing mRNA expression levels of inflammatory mediators in macrophage cell line RAW 264.7 after 2-hour incubation in control, 0.1 μg/mL recombinant HSPB4 (HSPB4 0.1), 1 μg/mL recombinant HSPB4 (HSPB4 1.0), in the presence or absence of 100 μg/mL DOPG (DOPG), including (FIG. 7A) IL1α, (FIG. 7B) IL1β, (FIG. 7C) IL6, and (FIG. 7D) TNFα, monitored by quantitative RT-PCR with GAPDH used as the housekeeping gene. Results represent the means±SEM of 3 separate experiments performed in duplicate; ***p<0.001 versus all other conditions except as indicated for which ξ<0.05 and ξξ<0.01.

FIGS. 8A-8B are bar graphs showing protein levels of TNFα secreted into the culture media of macrophage cell line RAW 264.7 after 2-hour incubation in control, (FIG. 8A) 2 μg/mL recombinant S100A9 (S100A9), (FIG. 8B) 0.1 μg/mL recombinant HSPB4 (HSPB4 0.1), or 1 μg/mL recombinant HSPB4 (HSPB4 1.0), in the presence or absence of 100 μg/mL DOPG (DOPG). Results represent the means±SEM of 3 separate experiments performed in duplicate; in panel A ***p<0.001 versus all other conditions; in panel B *p<0.05 versus the control and ξ<0.05 versus HSPB4 alone.

FIG. 9 is a bar graph showing ratios of phosphorylated NF-κB protein (pNFκB) versus non-phosphorylated NF-κB protein (NFκB) in RAW 264.7 macrophage cell line, treated with or without 2.5 μg/mL Pam₃CSK₄ (PAM) in the presence and absence of 100 μg/mL DOPG for 10 minutes. Results represent cumulative data as means±SEM from 3 separate experiments; ****p<0.05, p<0.001 relative to the control, §§§p<0.001 versus Pam alone.

FIGS. 10A-10C are bar graphs showing control mice, and contact irritant mouse ear edema model (i.e., ears treated with 12-O-tetra-decanoylphorbol 13-acetate (TPA)), treated with soy PG, with 1,25-dihydroxyvitamin D₃ (VitD), or the combination of soy PG and 1,25-dihydroxyvitamin D3 of their (FIG. 10A) ear thickness, (FIG. 10B) ear weight, and (FIG. 10C) number of CD45⁺ cells from formalin-fixed, paraffin-embedded ear biopsies. Results represent the means±SEM from 8-10 ears from 4-5 mice; *p<0.01 versus the untreated control; §p<0.01 versus TPA alone; # p<0.05 versus TPA+vitamin D; † p<0.01 versus TPA.

FIG. 11 is a bar graph showing ear thickness and ear weight of mice of contact irritant ear edema model (i.e., ears treated with 12-O-tetra-decanoylphorbol 13-acetate (TPA)), treated with vehicle (octanoic acid with added stearic acid), vehicle with 0.02% soy PG, or vehicle with 0.2% soy PG. Results represent means±SEM of 8-10 ears from 4-5 mice (6 ears from 3 mice served as the untreated controls); *p<0.05, **p<0.01 versus vehicle alone.

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx.+/−10%; in other embodiments the values may range in value either above or below the stated value in a range of approx.+/−5%; in other embodiments the values may range in value either above or below the stated value in a range of approx.+/−2%; in other embodiments the values may range in value either above or below the stated value in a range of approx.+/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “subject” or “individual” includes both humans, mammals (e.g., cats, dogs, horses, etc.), and other living species that are in need of treatment for inflammatory and/or autoimmune conditions/diseases. A living organism can be as simple as, for example, a single eukaryotic cell or as complex as a mammal. Further, a “composition” can include one or more chemical compounds, as described below.

The term “derivative” refers to a modification to the disclosed compounds including, but not limited to, hydrolysis, reduction, or oxidation products, of the disclosed compounds. Hydrolysis, reduction, and oxidation reactions are known in the art.

The term “functional derivative” refers to a derivative of the disclosed compounds that retains the function of the disclosed compound, at least in part. For instance, in the case of PG, a functional derivative of PG in the context of the present disclosure includes a derivative of PG which has the effect of modulating cells and molecules involved in the inflammatory response.

The term “carrier” or “excipient” refers to an organic or inorganic, natural or synthetic inactive ingredient in a formulation, with which one or more active ingredients are combined. In some embodiments, a carrier or an excipient is an inert substance added to a pharmaceutical composition to further facilitate administration of a compound, and/or does not cause significant irritation to an organism and does not abrogate the biological activity and properties of the administered compound.

The terms “effective amount” or “therapeutically effective amount” means a dosage sufficient to reduce, or inhibit inflammation of a disorder, disease, or condition being treated, or to otherwise provide a desired pharmacologic and/or physiologic effect. The precise dosage will vary according to a variety of factors such as subject-dependent variables (e.g., age, immune system health, etc.), the severity of the disease or disorder being treated, as well as the route of administration and the pharmacokinetics of the agent being administered.

The term “prevention” or “preventing” means to administer a composition to a subject or a system at risk for or having a predisposition for one or more symptom e.g., inflammation, caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

The term “inhibit,” “suppress,” “decrease,” “interfere,” and/or “reduce” (and like terms) generally refers to the act of reducing, either directly or indirectly, a function, activity, or behavior relative to the natural, expected, or average or relative to current conditions. It is understood that this is typically in relation to some standard or expected value, in other words it is relative, but that it is not always necessary for the standard or relative value to be referred to. For instance, something that inhibits, suppresses, decreases or reduces or interferes with inflammation associated with psoriasis might stop or slow the production, and/or secretion of pro-inflammatory cytokines by keratinocytes, and/or infiltration of immune cells involved in the pathogenesis of the condition at the site such as macrophages.

The term “increase,” “enhance,” “stimulate,” and/or “induce” (and like terms) generally refers to the act of improving or increasing, either directly or indirectly, a function or behavior relative to the natural, expected, or average or relative to current conditions. For instance, something that increases, stimulates, induces or enhances anti-inflammatory effects might induce the production, and/or secretion of anti-inflammatory cytokines, and/or infiltration of immune cells that mediate anti-inflammatory response such as Treg, or Th17 cells.

The terms “treat,” “treating,” and/or “treatment” are an approach for obtaining beneficial or desired clinical results. For purposes of embodiments of this disclosure, beneficial or desired clinical results include, but are not limited to, preventing the condition/disease from occurring in an animal that may be predisposed to the condition/disease but does not yet experience or exhibit symptoms of the disease (prophylactic treatment), alleviation of symptoms of the condition/disease, diminishment of extent of the condition/disease, stabilization (i.e., not worsening) of the condition/disease, preventing spread of the condition/disease, delaying or slowing of the condition/disease progression, amelioration or palliation of the condition/disease state, and combinations thereof, in addition, “treat”, “treating”, and “treatment” can also mean prolonging survival as compared to expected survival if not receiving treatment.

The term “parenteral administration”, means administration by any method other than through the digestive tract or non-invasive topical or regional routes. For example, parenteral administration may include administration to a patient intravenously, intradermally, intraperitoneally, intrapleurally, intratracheally, intramuscularly, subcutaneously, subjunctivally, by injection, and by infusion.

The term “topical administration”, means the non-invasive administration to the skin, orifices, or mucosa. Topical administrations can be administered locally, i.e., they are capable of providing a local effect in the region of application without systemic exposure. Topical formulations can provide systemic effect via adsorption into the blood stream of the individual. Topical administration can include, but is not limited to, cutaneous and transdermal administration, buccal administration, intranasal administration, intravaginal administration, intravesical administration, ophthalmic administration, and rectal administration.

The terms “bioactive agent” and “active agent”, used interchangeably, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

The terms “sufficient” and “effective”, used interchangeably, refer to an amount (e.g. mass, volume, dosage, concentration, and/or time period) needed to achieve one or more desired result(s).

The term “biocompatible”, refers to a material that along with any metabolites or degradation products thereof that are generally non-toxic to the recipient and do not cause any significant adverse effects to the recipient. Generally speaking, biocompatible materials are materials which do not elicit a significant inflammatory or immune response when administered to a patient.

The term “biodegradable”, generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

The term “pharmaceutically acceptable”, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration. A “pharmaceutically acceptable carrier”, refers to all components of a pharmaceutical formulation which facilitate the delivery of the composition in vivo. Pharmaceutically acceptable carriers include, but are not limited to, diluents, preservatives, binders, lubricants, disintegrators, swelling agents, fillers, stabilizers, and combinations thereof.

The term “molecular weight”, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

The term “consisting essentially of” limits the scope of a claim to the specified materials or steps “and those that do not materially affect the basic and novel characteristic(s)” of the claimed invention. In re Herz, 537 F.2d 549, 551-52, 190 USPQ 461, 463 (CCPA 1976).

### II. Compositions for Reducing Inflammation

Compositions including an effective amount of one or more phosphatidylglycerol (PG) for treating, or preventing inflammation are disclosed. Typically, administration of the composition is effective to reduce or inhibit inflammation in a subject in need thereof, for example inflammation related to psoriasis. In one embodiment, the composition contains 10 to 100 μg/mL in aqueous solution or 0.02 to 1% in a cream vehicle.

One embodiment provides compositions that are effective in reducing, or inhibiting quantities, and/or activities of one or more damage associated molecular patterns (DAMPs). Exemplary DAMPs inducing inflammatory effects include S100 proteins such as S100A1, S100A2, S100A3, S100A4, S100A5, S100A6, S100A7, S100A8, S100A9, S100A 10, S100A 11, S100A12, S100A13, S100A14, S100A15, S100A16, or combinations thereof. Some further exemplary DAMPs inducing inflammatory effects include small heat shock proteins including HSPB1, HSPB2, HSPB3, HSPB4, HSPB5, HSPB6, HSPB7, HSPB8, HSPB9, HSPB10, HSPB11, or combinations thereof.

Another embodiment provides compositions that cause direct or indirect reduction or inhibition of signaling through one or more receptors for damage associated molecular patterns (DAMPs) involved in the pathogenesis of the inflammatory conditions. Exemplary DAMP receptors include one or more members of the toll-like receptor (TLR) family such as TLR-1, TLR-2, TLR-4, TLR-6, or combinations thereof.

Still another embodiment provides compositions that cause direct or indirect reduction or inhibition of protein expression and/or secretion of pro-inflammatory cytokines from one or more cell types involved in the pathogenesis of psoriasis. These cells include, but not limited to epithelial cells such as keratinocytes, and immune cells infiltrated to the site such as macrophages, neutrophils, dendritic cells, T cells, and NKT cells. In some embodiments, the compositions lead to direct or indirect reduction or inhibition of protein expression and/or secretion of pro-inflammatory cytokines in these cells whilst having minimal, or no effects on the hyperproliferation of the keratinocytes associated with psoriasis. In further embodiments, the compositions lead to direct or indirect reduction or inhibition of protein expression and/or secretion of pro-inflammatory cytokines in these cells whilst reducing, or inhibiting the hyperproliferation of the keratinocytes associated with psoriasis. In some embodiments, the compositions can reduce or inhibit the mRNA, and/or protein levels of one or more pro-inflammatory cytokines such as IL1α, IL1β, IL6 and TNFα. In some embodiments, the compositions lead to direct or indirect inhibition of protein expression, and/or secretion of one or more pro-inflammatory cytokines such as IL1α, IL1β, IL6 and TNFα in both keratinocytes and macrophages.

Yet another embodiment provides compositions that cause direct or indirect reduction or inhibition of one or more protein transcription factors involved in the pathogenesis of psoriasis. In a specific embodiment, the compositions are effective in reduce, or inhibit inflammation associated with increased activity of the protein transcription factor NF-κB.

A. Phosphatidylglycerol

Typically, the compositions include one or more phosphatidylglycerol (PG) molecules, or functional derivatives thereof. Phosphatidylglycerol is a ubiquitous lipid that can be the main component of some bacterial membranes, and it is found also in membranes of plants and animals where it appears to perform specific functions. The charge on the phosphate group means that it is an anionic lipid at neutral pH.

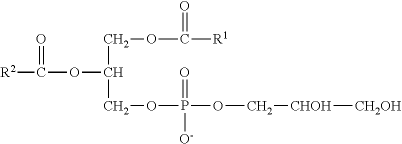

Formula I General chemical structure of a phosphatidyl glycerol where R¹ and R² are fatty acid side chains.

In some embodiments, the phosphatidylglycerol species contains one or more monounsaturated fatty acids. In other embodiments, the phosphatidylglycerol species contains one or more polyunsaturated fatty acids. In some embodiments, the PG is egg-derived PG, or soy PG. Egg-derived PG exhibits the following fatty acid composition (with the first number representing the total number of carbon atoms in the fatty acid and the second number, the number of double bonds): 16:0 (34%) 16:1 (2%), 18:0 (11%), 18:1 (32%), 18:2 (18%) and 20:4 (3%). Soy PG is a PG mixture with a large percentage of polyunsaturated fatty acids, composed of 16:0 (17%), 18:0 (6%), 18:1 (13%), 18:2 (59%), and 18:3 (5%). Soy PG is efficacious at inhibiting rapidly proliferating keratinocytes. In a preferred embodiment, the composition for reducing, or inhibiting inflammation comprises soy PG.

In one embodiment, the PG is dioleoyl-PG (two 18-carbon fatty acids with one double bond each, denoted as 18:1/18:1). Exemplary PGs also include palmitoyl-arachidonyl-PG (16:0/20:4), palmitoyl-linoleoyl-PG (16:0/18:2), dilinoleoyl-PG (18:2/18:2), palmitoyl-oleoyl-PG (16:0/18:1), dioleoyl-PG (18:1/18:1), and dihexanoylphosphatidylglycerol (DHPG), dipalmitoylphosphatidylglycerol (DPPG), di stearoylphosphatidylglycerol (DSPG), palmitoyl-oleoylphosphatidylglycerol (POPG).

In some embodiments, the R¹ and R² fatty acid chains are independently selected from any common fatty acid molecules such as myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, and docosahexaenoic acid.

In some embodiments, the R¹ and R² fatty acid chains are independently fatty acid molecules with 4-28 carbons, preferably 13-21 carbons, with 1-8 double bonds, preferably 1 double bond, 2 double bonds, 3 double bonds or 4 double bonds, in cis and/or trans configuration, or any combinations thereof. \

B. Additional Therapeutic, Prophylactic or Diagnostic Agents

The disclosed compositions can be administered in combination or alternation with one or more additional therapeutic, diagnostic, and/or prophylactic agents. For example, one or more additional therapeutic, diagnostic, and/or prophylactic agents can reduce, or alleviate itching, burning, soreness, and dryness in conditions such as psoriasis. One embodiment provides administering the disclosed compositions in combination or alternation with one or more additional therapeutic, diagnostic, and/or prophylactic agents to reduce or inhibit red patches of skin, silvery scaling, scaling spots, or scarring in psoriasis. In some embodiments, the composition may contain one or more additional compounds to further relieve inflammation. In some embodiments, the disclosed composition is used in combination with one or more anti-angiogenesis agents.

The amount of a second therapeutic generally depends on the severity of the condition to be treated. Specific dosages can be readily determined by those of skill in the art. See Ansel, Howard C. et al. Pharmaceutical Dosage Forms and Drug Delivery Systems (6^{th} ed.) Williams and Wilkins, Malvern, Pa. (1995).

In some cases, one or more additional active agents may be dispersed in, or otherwise associated with particles in the formulation. In certain embodiments, one or more additional active agents may also be dissolved or suspended in the pharmaceutically acceptable carrier.

In some cases, the active agent is a diagnostic agent imaging or otherwise assessing the inflammatory conditions. Exemplary diagnostic agents include paramagnetic molecules, fluorescent compounds, magnetic molecules, and radionuclides, x-ray imaging agents, and contrast media.

In some embodiments, the disclosed composition is used in combination with one or more topical medicines. Topical medicines are applied directly to the skin to treat scaly, itchy rashes due to psoriasis. Available in creams, gels, lotions, shampoos, sprays or ointments, these drugs are available over-the-counter (OTC) and by prescription. OTC ones include salicylic acid, which helps lift and peel scales, and coal tar, which may slow rapid cell growth of scales and ease itching and inflammation. Prescription topicals contain corticosteroids and/or vitamin derivatives. Common prescription ones include calcitriol, a naturally occurring form of vitamin D3; calcipotriene, a synthetic form of vitamin D3; calcipotriene combined with the corticosteroid betamethasone dipropionate; tazarotene (a vitamin-A derivative); and anthralin, a synthetic form of chrysarobin, a substance derived from the South American araroba tree.

In some embodiments, the disclosed composition is used in combination with one or more anti-inflammatory agents. Anti-inflammatory agents reduce inflammation and include steroidal and non-steroidal drugs. Suitable steroidal active agents include glucocorticoids, progestins, mineralocorticoids, and corticosteroids. Small molecule non-steroidal anti-inflammatory drugs (NSAIDs) include, but are not limited to, ibuprofen, naproxen, aspirin, ketoprofen, nepafenac, diclofenac, indomethacin, piroxicam, meloxicam, sulindac, methotrexate, leflunomide, hydroxychloroquine, and sulfasalazine. Small molecule steroidal anti-inflammatories include, but are not limited to, prednisone, dexamethasone, cortisone, loteprendol, triamcinolone acetonide, fluocinolone acetonide, fluorometholone, and fluticasone.

Exemplary immune-modulating drugs include cyclosporine, tacrolimus and rapamycin. In some embodiments, anti-inflammatory agents are biologic drugs that block the action of one or more immune cell types such as T cells, or block proteins in the immune system, such as tumor necrosis factor-alpha (TNF-alpha), interleukin 17-A, interleukins 12 and 23.

Inflammatory disorder, and/or autoimmune diseases can predispose to infectious diseases. For example, streptococcal infection is strongly associated with the development of guttate psoriasis, and/or chronic plaque psoriasis. Thus, in some embodiments, the disclosed composition is used in combination with one or more antimicrobial agents.

An antimicrobial agent is a substance that kills or inhibits the growth of microbes such as bacteria, fungi, viruses, or parasites. Antimicrobial agents include antiviral agents, antibacterial agents, antiparasitic agents, and anti-fungal agents. Representative antiviral agents include ganciclovir and acyclovir. Representative antibiotic agents include aminoglycosides such as streptomycin, amikacin, gentamicin, and tobramycin, ansamycins such as geldanamycin and herbimycin, carbacephems, carbapenems, cephalosporins, glycopeptides such as vancomycin, teicoplanin, and telavancin, lincosamides, lipopeptides such as daptomycin, macrolides such as azithromycin, clarithromycin, dirithromycin, and erythromycin, monobactams, nitrofurans, penicillins, polypeptides such as bacitracin, colistin and polymyxin B, quinolones, sulfonamides, and tetracyclines. Other exemplary antimicrobial agents include iodine, silver compounds, moxifloxacin, ciprofloxacin, levofloxacin, cefazolin, tigecycline, gentamycin, ceftazidime, ofloxacin, gatifloxacin, amphotericin, voriconazole, natamycin.

In some embodiments, one or more therapeutic, prophylactic or diagnostic agent is administered prior to, in conjunction with, subsequent to, or alternation with treatment using the disclosed compositions.

In the case of pharmaceutical compositions for the treatment of ocular diseases, the formulation may contain one or more ophthalmic drugs to treat, prevent or diagnose a disease or disorder of the eye. Non-limiting examples of ophthalmic drugs include anti-glaucoma agents, anti-angiogenesis agents, anti-infective agents, anti-inflammatory agents, an analgesic, a local anesthetic, growth factors, immunosuppressant agents, anti-allergic agents, an anti-oxidant, a cytokine, and combinations thereof. Representative anti-glaucoma agents include prostaglandin analogs (such as travoprost, bimatoprost, and latanoprost), beta-andrenergic receptor antagonists (such as timolol, betaxolol, levobetaxolol, and carteolol), alpha-2 adrenergic receptor agonists (such as brimonidine and apraclonidine), carbonic anhydrase inhibitors (such as brinzolamide, acetazolamine, and dorzolamide), miotics (i.e., parasympathomimetics, such as pilocarpine and ecothiopate), seretonergics muscarinics, dopaminergic agonists, and adrenergic agonists (such as apraclonidine and brimonidine). Representative anti-angiogenesis agents include, but are not limited to, antibodies to vascular endothelial growth factor (VEGF) such as bevacizumab (AVASTIN®) and rhuFAb V2 (ranibizumab, LUCENTIS®), and other anti-VEGF compounds including aflibercept (EYLEA®); MACUGEN® (pegaptanim sodium, anti-VEGF aptamer or EYE001) (Eyetech Pharmaceuticals); pigment epithelium derived factor(s) (PEDF); COX-2 inhibitors such as celecoxib (CELEBREX®) and rofecoxib (VIOXX®); interferon alpha; interleukin-12 (IL-12); thalidomide (THALOMID®) and derivatives thereof such as lenalidomide (REVLIMID®); squalamine; endostatin; angiostatin; ribozyme inhibitors such as ANGIOZYME® (Sirna Therapeutics); multifunctional antiangiogenic agents such as NEOVASTAT® (AE-941) (Aeterna Laboratories, Quebec City, Canada); receptor tyrosine kinase (RTK) inhibitors such as sunitinib (SUTENT®); tyrosine kinase inhibitors such as sorafenib (Nexavar®) and erlotinib (Tarceva®); antibodies to the epidermal grown factor receptor such as panitumumab (VECTIBIX®) and cetuximab (ERBITUX®), as well as other anti-angiogenesis agents known in the art.

C. Kits

Medical kits are also disclosed. Typically, the described compositions are prepared using a pharmaceutically acceptable carrier composed of materials that are considered safe and effective and may be administered to an individual without causing undesirable biological side effects or unwanted interactions. A kit can include one or more of the compounds or compositions described. For example, a kit can be a container that includes a compound of phosphatidylglycerol, or a mixture of PGs such as soy PG. A kit can further include one or more topical medications (e.g., hydrocortisone). A kit for ophthalmic applications can be ready for administration in an eye dropper bottle, optionally further includes lubricating eye drops or artificial tears. A kit can additionally include directions for use of the kit (e.g., instructions for treating a subject).

D. Excipients

Formulations and pharmaceutical compositions containing an effective amount of the composition in a pharmaceutical carrier appropriate for administration to a subject in need thereof to reduce or inhibit inflammation are provided. The formulations are designed for administration topically (e.g., to a mucosal surface such as the mouth, conjunctiva, intranasal, intravaginally, etc.). It may also be possible to administer parenterally (e.g., by intramuscular, intraperitoneal, intravenous (IV) or subcutaneous injection or infusion). The compositions designed to be administered locally or systemically.

Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

The compositions can be formulated for immediate release, extended release, or modified release or PG. A delayed release dosage form is one that releases a drug (or drugs) at a time other than promptly after administration. An extended release dosage form is one that allows at least a twofold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients.

Generally, pharmaceutically acceptable salts can be prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, M I D, 2000, p. 704. Examples of ophthalmic drugs sometimes administered in the form of a pharmaceutically acceptable salt include timolol maleate, brimonidine tartrate, and sodium diclofenac.

In some embodiments, the active agent, PG, is incorporated into or encapsulated by a nanoparticle, microparticle, micelle, synthetic lipoprotein particle, liposome, or carbon nanotube. For example, the compositions can be incorporated into a vehicle such as polymeric microparticles which provide controlled release of the active agent. In some embodiments, release of the drug(s) is controlled by diffusion of the active agent out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof.

The active agent can be incorporated into or prepared from materials which are insoluble in aqueous solution or slowly soluble in aqueous solution, but are capable of degrading within the site of action including enzymatic degradation, surfactant action, and/or mechanical erosion. As used herein, the term “slowly soluble in water” refers to materials that are not dissolved in water within a period of 30 minutes. Preferred examples include fats, fatty substances, waxes, waxlike substances and mixtures thereof. Suitable fats and fatty substances include fatty alcohols (such as lauryl, myristyl stearyl, cetyl or cetostearyl alcohol), fatty acids and derivatives, including, but not limited to, fatty acid esters, fatty acid glycerides (mono-, di- and tri-glycerides), and hydrogenated fats. Specific examples include, but are not limited to, hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes.

Specific examples of waxes include beeswax, glycowax, castor wax, carnauba wax, paraffins and candelilla wax. As used herein, a wax-like material is defined as any material which is normally solid at room temperature and has a melting point of from about 30 to 300° C.

1. Solutions, Gels, Ointments and Suspension

Numerous parenteral or topical formulations are known and available. Solutions can be the sterile filtered, concentrated or diluted with water, buffered saline, or an equivalent, formed into a gel with a polysaccharide such as alginate or hyaluronic acid, polyvinyl pyrrole, or ointment such as petrolatum or mineral oil, or emulsified with lipid or oil.

Emulsions are generally dispersions of oily droplets in an aqueous phase. There should be no evidence of breaking or coalescence.

Suspensions contain solid particles dispersed in a liquid vehicle; they must be homogeneous when shaken gently and remain sufficiently dispersed to enable the correct dose to be removed from the container. A sediment may occur, but this should disperse readily when the container is shaken, and the size of the dispersed particles should be controlled. For ophthalmic application, the active ingredient and any other suspended material must be reduced to a particle size small enough to prevent irritation and damage to the cornea.

Ointments are sterile, homogeneous, semi-solid preparations intended for application to the surface, such as the skin, the conjunctiva or the eyelids. They are usually prepared from non-aqueous bases, e.g., soft paraffin (Vaseline), liquid paraffin, and wool fat. They may contain suitable additives, such as antimicrobial agents, antioxidants, and stabilizing agents.

The composition can be formulated for parenteral delivery, such as injection or infusion, in the form of a solution or suspension, or a powder. The formulation can be administered via any route, such as, the blood stream or directly to the organ or tissue to be treated.

The composition can also be formulated for topical delivery (e.g., to a mucosal surface such as the mouth, conjunctiva, intranasal, intravaginally, etc.). The particles may be provided in a lyophilized or dried form in a unit dosage form, for suspension at the time of injection. These may be provided in a kit with an appropriate amount of diluent such as sterile water or buffered solution.

The formulations can be prepared as aqueous compositions using techniques known in the art. The compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the compounds or nanoparticles can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s) or nanoparticles.

The formulation is typically buffered to a pH of between 3 and 8 for parenteral or topical administration upon reconstitution. Suitable buffers include, but are not limited to, phosphate buffers, acetate buffers, and citrate buffers. Ideally, the pH of ophthalmic drops should be equivalent to that of tear fluid, which is 7.4. However, the decision to add a buffering agent should be based on stability considerations. The pH selected should be the optimum for both stability of the active pharmaceutical ingredient and physiological tolerance. If a buffer system is used, it must not cause precipitation or deterioration of the active ingredient. The influence on the lachrymal flow should also be taken into account.

Although solutions with the same pH as lacrimal fluid (7.4) are ideal, the outer surfaces of the eye tolerate a larger range, 3.5 to 8.5. The normal useful range to prevent corneal damage is 6.5 to 8.5. The final pH of the solution is often a compromise, because many ophthalmic drugs have limited solubility and stability at the desired pH of 7.4. Solutions that are isotonic with tears are preferred. An amount equivalent to 0.9% NaCl is ideal for comfort and should be used when possible. The eye can tolerate tonicities within the equivalent range of 0.6-2% NaCl without discomfort. The most widely used ophthalmic buffer solutions are boric acid vehicle and Sorensen's modified phosphate buffer. The boric acid vehicle is a 1.9% solution of boric acid in purified water or preferably sterile water. There are times when hypertonic ophthalmic solutions are necessary therapeutically, or when the addition of an auxiliary agent required for reasons of stability supersedes the need for isotonicity. A hypotonic ophthalmic solution will require the addition of a substance (tonicity adjusting agent) to attain the proper tonicity range.

Water soluble polymers are often used in formulations for parenteral or topical administration. Suitable water-soluble polymers include, but are not limited to, polyvinylpyrrolidone, dextran, carboxymethylcellulose, and polyethylene glycol.

Sterile injectable solutions can be prepared by incorporating the compound or nanoparticles in the required amount in the appropriate solvent or dispersion medium with one or more of the excipients listed above, as required, followed by filtered sterilization. Generally, dispersions are prepared by incorporating the various sterilized compositions into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the compound or nanoparticle plus any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral or topical administration are preferably in the form of a sterile aqueous solution or suspension of particles formed from one or more polymer-drug conjugates. Acceptable solvents include, for example, water, Ringer's solution, phosphate buffered saline (PBS), and isotonic sodium chloride solution. The formulation may also be a sterile solution, suspension, or emulsion in a nontoxic, acceptable diluent or solvent such as 1,3-butanediol.

In some instances, the formulation is distributed or packaged in a liquid form. Alternatively, formulations for parenteral or topical administration can be packed as a solid, obtained, for example by lyophilization of a suitable liquid formulation. The solid can be reconstituted with an appropriate carrier or diluent prior to administration.

Solutions, suspensions, or emulsions for parenteral or topical administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for parenteral or topical administration may also contain one or more tonicity agents to adjust the isotonic range of the formulation. Suitable tonicity agents are well known in the art. Examples include glycerin, mannitol, sorbitol, sodium chloride, and other electrolytes.

Solutions, suspensions, or emulsions for parenteral or topical administration may also contain one or more preservatives to prevent bacterial contamination of the tropical preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for parenteral administration may also contain one or more excipients known art, such as dispersing agents, wetting agents, and suspending agents.

In some forms, the carrier can comprise a cream, paste, fluid, coating, paint, spray, detergent, or a combination thereof. In some embodiments, the disclosed compositions can be used in any forms that come to direct and/or indirect contact with the skin. For example, in body cream/paste/lotion/gel, body soap, skin wash, laundry detergent, skin spray, wound cleanser, wound covering, shampoo, shower gel, facial wash, facial cream, or facial soap.

In some embodiments, the compositions can further comprise a carrier that enhances the delivery of the compositions to the cells of interest, e.g., keratinocytes and macrophages associated with the pathogenesis of psoriasis. A carrier can be barrier-disrupting agent or a penetration-enhancing vehicle. Exemplary penetration-enhancing vehicles include a humectant (e.g., glycols, glycerols), powder, (e.g., clays, shake lotions), oil/water (O/W) emulsion (e.g., aqueous creams), water/oil emulsion (e.g., oily creams), emulsifying base (e.g., anhydrous lipid and O/W emulsifiers), absorption base (e.g., anhydrous lipid and W/O emulsifiers), lipophilic (e.g., fats, waxes, oils, silicones), salicylic acid, urea phospholipase A2, phosphatidylcholine dependent phospholipase C, ethanol, acetone, detergents, bases, propylene glycol, pyrriolidones, dimethylacetamide, dimethylformamide, dimethylsulfoxide, alkyl sulfoxide, phosphine oxide, surfactants and caprolactams such as azone, amines and amides, alkyl N,N-distributed-amino acetates, decylmethylsulfoxide, pyrrolidones, pirotiodecane (HPE-101), benzlyalkonium, benzylalkonium chloride polymers, silicone based polymers, fatty acids, cyclic ureas, terpenes, liposomes, cyclodextrins, and combinations thereof.

### III. Methods of Reducing Inflammation

Methods of using the compositions to treat or prevent inflammation in a subject are provided.

Methods typically include administering a subject in a need thereof an effective amount of a composition including one or more phosphatidylglycerol, or functional derivatives thereof. In some embodiments, the subject is in need of treatment of an inflammatory disease such as a skin inflammatory disease. In one embodiment, the subject has been diagnosed with psoriasis. In a further embodiment, the subject has inflammation in the eye.

Methods of using the compositions to treat or prevent inflammatory conditions caused by one or more damage associated molecular patterns (DAMPs) are described. Exemplary DAMPs inducing inflammatory effects include S100 proteins such as S100A1, S100A2, S100A3, S100A4, S100A5, S100A6, S100A7, S100A8, S100A9, S100A 10, S100A 11, S100A12, S100A13, S100A14, S100A15, S100A16, or combinations thereof. Some further exemplary DAMPs inducing inflammatory effects include small heat shock proteins including HSPB1, HSPB2, HSPB3, HSPB4, HSPB5, HSPB6, HSPB7, HSPB8, HSPB9, HSPB10, HSPB11, or combinations thereof. In a specific embodiment, the inflammatory conditions are caused by S100A9. S100A12 showed the closest association with disease activity and therapeutic response in psoriasis (Wilsmann-Theis D et al., J Eur Acad Dermatol Venereol. 30(7):1165-70 (2016)). In a further embodiment, the inflammatory conditions are caused by S100A12, and/or one or more further DAMPs. In one embodiment, the inflammatory conditions are caused by HSPB4. In some embodiments, the disclosed compositions are administered to a subject in an effective amount to reduce, or inhibit activities of one or more DAMPs. In some embodiments, the disclosed compositions suppress the inflammation mediated directly, and/or indirectly by one or more DAMPs, by 10%, 20%, 30%, 40%, 50%, 60%, 70%, 80%, 90%, or more than 90%. Such reduction can be measured by downstream effectors such as inflammatory mediators including IL1α, IL1β, IL6, TNFα, or activation status of protein transcription factors such as NF-κB, or combinations thereof.

Methods of using the compositions to treat, or prevent inflammatory conditions caused by activation through one or more receptors for damage associated molecular patterns (DAMPs) are described. Exemplary DAMP receptors include one or more members of the toll-like receptor (TLR) family such as TLR-1, TLR-2, TLR-4, TLR-6, or combinations thereof. In some embodiments, the disclosed compositions are suitable for skin inflammation caused by activation through TLR-2. In some embodiments, the disclosed compositions are administered to a subject in an effective amount to reduce, or inhibit activation/signaling though one or more receptors for DAMPs involved in the pathogenesis of the inflammatory conditions such as psoriasis. In some embodiments, the disclosed compositions suppress the inflammation mediated directly, and/or indirectly though one or more receptors for DAMPs, by 10%, 20%, 30%, 40%, 50%, 60%, 70%, 80%, 90%, or more than 90%. Such reduction can be measured by downstream effectors such as inflammatory mediators including IL1α, IL1β, IL6, TNFα, or activation status of protein transcription factors such as NF-κβ, or combinations thereof.

Methods of using the compositions to treat, or prevent inflammatory conditions caused by one or more pro-inflammatory cytokines are described. Exemplary pro-inflammatory cytokines include IL1α, IL1β, IL6, and TNFα. In some embodiments, compositions are administered to a subject in an effective amount to reduce the expression levels of one or more pro-inflammatory cytokines, reduce the activities of one or more pro-inflammatory cytokines, reduce the secretion of one or more pro-inflammatory cytokines, reduce the ratio of pro-inflammatory cytokines to anti-inflammatory cytokines, or a combination thereof. Typically, the disclosed compositions are effective in reducing the activity and/or quantity of one or more pro-inflammatory cytokines in one or more cell types, for example, in both keratinocytes and macrophages. In some embodiments, the disclosed compositions lead to direct, and/or indirect reduction of one or more pro-inflammatory cytokines by 5%, 10%, 20%, 30%, 40%, 50%, 60%, 70%, 80%, 90%, or more than 90%.

Methods of using the compositions to treat, or prevent inflammatory conditions caused by activation of one or more protein transcription factors involved in the pathogenesis of the inflammatory condition are described. For example, NF-κB is a crucial mediator involved in the pathogenesis of psoriasis. Thus, in some embodiments, the compositions are administered to a subject in an effective amount to reduce, or inhibit the activity of one or more protein transcription factors involved in the pathogenesis of the inflammatory conditions such as psoriasis. In a specific embodiment, the protein transcription factor is NF-κB.

One study identified the HSPB4/TLR2/NF-κB axis in macrophage as a therapeutic target for sterile inflammation of the cornea (Oh J Y et al., EMBO molecular medicine 4, 435-448 (2012)). Thus, in some embodiments, the compositions are administered to a subject in an effective amount to modulate activity of one or more molecules that lead direct, and/or indirect reduction, or inhibition of the HSPB4/TLR2/NF-κB axis, preferably in macrophages.

A. Treatment Regimen and Dosage

1. Topical Formulations

One embodiment provides compositions and methods of use thereof developed for topical application to the skin, for treatment of skin inflammatory disease, particularly chronic skin inflammatory disease e.g., psoriasis. Quantities of the present compositions which are typically applied per application are, in mg composition/cm² skin, from about 0.1 mg/cm² to about 100 mg/cm², depending on the severity of the condition to be treated. Alternatively, the PG in the final composition may be between about 1 μM and about 1 mM, preferably between about 1 μM and about 100 μM, and most preferably between about 10 μM and about 200 μM. Alternatively, the concentration of PG can be 1 μg/mL to 500 μg/mL, 20 to 50 μg/mL, 75 to 100 μg/mL

Application to the skin is preferably practiced by applying a composition in the form of a skin lotion, cream, gel, emulsion, spray, shampoo, conditioner, cosmetic, lipstick, foundation, nail polish, or the like which is intended to be left on the skin for some aesthetic, prophylactic, therapeutic or other benefit (i.e., a “leave-on” composition). After applying the composition to the skin, it is preferably left on the skin for a period of at least about 15 minutes, more preferably at least about 30 minutes, even more preferably at least about 1 hour, most preferably for at least several hours, e.g., up to about 12 hours. Any part of the external portion of the face, body, and/or nails can be treated, e.g., face, lips, under-eye area, eyelids, scalp, neck, torso, arms, hands, legs, feet, fingernails, toenails, etc.

Another approach to ensure a continuous exposure of the skin to at least a minimum level of the disclosed compositions is to apply the compositions by use of a patch applied, e.g., to the area of the skin with inflammation. Such an approach is particularly useful for problem skin areas needing more intensive treatment. The patch can be occlusive, semi-occlusive or non-occlusive. The composition containing one or more phosphatidylglycerol molecules can be contained within the patch or be applied to the skin prior to application of the patch. The patch can also include additional compounds to treat the inflamed area for inflammation and/or other symptoms. The patch is preferably left on the skin for a period of at least about 15 minutes, more preferably at least about 30 minutes, even more preferably at least about 1 hour, most preferably at night as a form of night therapy.

in some forms of treating skin disorders, the term “effective amount” or “therapeutically effective amount” means a dosage sufficient to treat, inhibit, or alleviate one or more inflammatory symptoms of the disorder being treated or to otherwise provide a desired pharmacologic and/or physiologic effect.

2. Ophthalmic Formulations

Compositions and methods of use thereof have also been developed for topical application to the eye, for the treatment of inflammation associated with ocular diseases and injuries. The method involves the management of a specifically formulated fluid containing one or more phosphatidylglycerol, or functional derivatives thereof applied directly to the eye(s), preferably as a liquid ophthalmic solution, much like a common liquid eye drops, lubricant or gel. The formulation delivered to the surface of the eye can alleviate or prevent at inflammation of the eye(s) associated with a number of ocular injuries and diseases, including chronic dry eye disease, Sjogren's syndrome, burns or injuries, corneal neovascular disorders, retinitis pigmentosa, macular degeneration, retinal ischemia, and corneal opacities (including corneal haze). For ophthalmological applications, two to three drops of solution will be administered once or twice daily, or as often as needed.

Topical administration is employed mostly in the form of eye drops, ointments, gels, or emulsions, to treat eye diseases. Topical application has remained the most preferred method due to the ease of administration and low cost. For most of the topically applied drugs, the site of action is usually different layers of the cornea, conjunctiva, sclera, and the other tissues of the anterior segment such as the iris and ciliary body (anterior uvea). Upon administration, precorneal factors and anatomical barriers negatively affect the bioavailability of topical formulations. Precorneal factors include solution drainage, blinking, tear film, tear turn over, and induced lacrimation. Human tear volume is estimated to be 7 μL, and the cul-de-sac can transiently contain around 30 μL of fluid. However, tear film displays a rapid restoration time of 2-3 min, and most of the topically applied solutions are washed away within 15-30 s after instillation. Considering all the precorneal factors, contact time with the absorptive membranes is low, which is considered to be the primary reason for less than 5% of the applied dose reaching the intraocular tissues (USP. USP 36-NF 31, Ophthalmic Ointments 771. Rockville, MID: USP; 2013).

The appropriate amounts are determined on an individual basis, measuring response to treatment over time. The concentration and dosage (number of times per day of amount of formulation for period of time) will vary depending on the condition to be treated, the severity of the condition, and the inclusion of other therapeutic, prophylactic or diagnostic agents. Thus, it is not possible to specify an exact amount for every composition. However, an appropriate amount can be determined by one of ordinary skill in the art using only routine experimentation given the teachings herein.

In the case of sustained or controlled release formulations, ointments, implants or injections into the site of treatment, the dosages will be modified to deliver a therapeutically equivalent amount.

One embodiment provides colloidal dosage forms for ocular drug delivery. These dosage forms include liposomes, nanoparticles, microemulsions, nanoemulsions, etc. Advantages of colloidal dosage forms include sustained and controlled release of the drug at the targeted site, reduced frequency of administration, and ability to overcome blood-ocular barriers.

B. Combination Therapies and Procedures

In some embodiments, the compositions are administered prior to, in conjunction with, subsequent to, or alternation with a further procedure. For example, for persistent, difficult-to-treat cases of psoriasis, many doctors recommend light therapy. In phototherapy or light therapy, the skin is regularly exposed to ultraviolet light. One of the most effective treatments is PUVA (the drug psoralen combined with ultraviolet A, or UVA, light). However, this form of therapy is used far less often today, because it has been shown to increase the risk of developing skin cancer. Some doctors may prescribe ultraviolet B light (UVB) treatment using a light box alone or with other therapies such as coal tar. A more targeted ultraviolet light treatment, called narrow-band UVB therapy, is less carcinogenic than PUVA but almost as effective. Thus, in some embodiments, the light therapy is administered prior to, in conjunction with, subsequent to, or alternation with treatment using the disclosed compositions.

In some embodiments, the compositions are administered prior to, in conjunction with, subsequent to, or alternation with a systemic medication. Some doctors prescribe oral drugs to treat psoriasis. Some of these medications affect the immune system. One such medication, methotrexate (also used as a chemotherapy drug for cancer and for various forms of arthritis), can produce dramatic clearing of the psoriasis lesions. Another medication of this type is cyclosporine. Oral retinoids, compounds with vitamin-A-like properties, can be mildly helpful to people with severe psoriasis. Newer treatments for people with severe psoriasis and psoriatic arthritis are now available. Several “biologic” drugs, which are made from human or animal proteins, focus on controlling the body's immune response. Exemplary biologic drugs include adalimumab (HUMIRA®), etanercept (ENBREL®), etanercept-szzs (ERELZI®), ixekizumab (TALZ®), secukinumab (COSENTYX®), and ustekinumab (STELARA®). Therefore, in some embodiments, systemic oral medications are administered prior to, in conjunction with, subsequent to, or alternation with treatment using the disclosed compositions.

In the case of ophthalmic application, the compositions are administered prior to, in conjunction with, subsequent to, or alternation with a further procedure such as a surgical procedure. For example, ocular inflammation is common amongst patients who have just undergone cataract surgery. Thus, the compositions are administered to reduce, or inhibit the ocular inflammation after cataract surgery.

C. Conditions to be Treated

The disclosed compositions are particularly suited to treat an inflammatory response or autoimmune disorder in a subject. For example, the disclosed methods can be used to prophylactically or therapeutically inhibit, reduce, alleviate, or permanently reverse inflammation of an inflammatory response or autoimmune disorder. An inflammatory response or autoimmune disorder can be inhibited or reduced in a subject by administering to the subject an effective amount of the disclosed compositions.

Representative inflammatory responses and autoimmune diseases that can be inhibited or treated include, but are not limited to, rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Bechet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis.

In some embodiments, the compositions and methods of treatment thereof are particularly useful in the context of inflammatory diseases of the skin. The compositions are suitable for management or treatment of psoriasis.

Psoriasis signs and symptoms can vary from person to person but may include one or more of the following: red patches of skin covered with silvery scales, small scaling spots (commonly seen in children), dry, cracked skin that may bleed, itching, burning or soreness, thickened, pitted or ridged nails, swollen and stiff joints. Psoriasis patches can range from a few spots of dandruff-like scaling to major eruptions that cover large areas. Most types of psoriasis go through cycles, flaring for a few weeks or months, then subsiding for a time or even going into complete remission.

Plaque psoriasis is the most common form of the disease and appears as raised, red patches covered with a silvery white buildup of dead skin cells or scale. These patches or plaques most often appear on the scalp, knees, elbows, and lower back. They are often itchy and painful, and they can crack and bleed. Guttate psoriasis is a form of psoriasis that appears as small, dot-like lesions. Guttate psoriasis often starts in childhood or young adulthood, and can be triggered by a strep infection. This is the second-most common type of psoriasis, after plaque psoriasis. About 10 percent of people who get psoriasis develop guttate psoriasis. Inverse psoriasis shows up as very red lesions in body folds, such as behind the knee, under the arm or in the groin. It may appear smooth and shiny. Many people have another type of psoriasis elsewhere on the body at the same time. Pustular psoriasis in characterized by white pustules (blisters of noninfectious pus) surrounded by red skin. The pus consists of white blood cells. It is not an infection, nor is it contagious. Pustular psoriasis can occur on any part of the body, but occurs most often on the hands or feet. Erythrodermic psoriasis is a particularly severe form of psoriasis that leads to widespread, fiery redness over most of the body. It can cause severe itching and pain, and make the skin come off in sheets. It is rare, occurring in 3 percent of people who have psoriasis during their life time. It generally appears on people who have unstable plaque psoriasis.

In some embodiments, the disclosed compositions are suitable for management of plaque psoriasis, nail psoriasis, scalp psoriasis, guttate psoriasis, inverse psoriasis, pustular psoriasis, erythrodermic psoriasis, or psoriatic arthritis.

In some embodiments, the disclosed compositions are suitable for reducing, or inhibiting inflammation associated with ocular diseases and/or injuries. Exemplary ocular diseases and/or injuries include uveitis, chronic dry eye disease, Sjogren's syndrome, burns or injuries, corneal neovascular disorders, retinitis pigmentosa, macular degeneration, retinal ischemia, and corneal opacities (including corneal haze).

In some embodiments the subject to be treated is a human. In some embodiments the subject to be treated are mammals such as horses, dogs and cats. All the methods described can include the step of identifying and selecting a subject in need of treatment, or a subject who would benefit from administration with the described compositions.

D. Controls

The effect of the described composition can be compared to a control. Suitable controls are known in the art and include, for example, an untreated subject, or a placebo-treated subject. In some embodiments, an untreated control subject suffers from, the same disease or condition as the treated subject e.g., psoriasis, preferably having a similar degree of severity. In some embodiments, a suitable control is an area having the same inflammatory condition as the area being treated on the same subject. For example, a patient with psoriatic skin on both feet can treat one foot with the compositions, whilst use the other as a control.

### EXAMPLES

### Example 1: DOPG Inhibits Keratinocyte Inflammatory Mediator Expression in Response to Pam₃CSK₄

### Methods and Materials

Keratinocyte Preparation and Cell Culture

Primary cultures of mouse epidermal keratinocytes were prepared from ICR strain CD-1 outbred neonatal mice 1-3 days of age as described in detail in (Bollag W B, et al., J Invest Derm 100 240-246 (1993); Griner R, et al., J Biol Chem, 274 4663-4670 (1999)). All procedures were approved by the Institutional Animal Care and Use Committee. RAW264.7 cells, a mouse macrophage-derived cell line, were kindly provided by Drs. Qing Zhong and Carlos Isales (Augusta University) and were cultured in DMEM containing 10% fetal bovine serum and 1% penicillin and streptomycin.

Contact Irritant Ear Edema Mouse In Vivo Model—

Experiments were performed as described in (27). Briefly, the ears of three to five male ICR CD-1 outbred mice [25-30 g; 5-6 weeks of age from Harlan Laboratories (Indianapolis, Ind.)] were treated with acetone or TPA (0.03% in acetone), and at 1 h and 4 h after TPA, the appropriate vehicle or the treatment in vehicle was applied to each ear. Ear thickness was measured using a caliper both before and at approximately 18-20 h after TPA treatment prior to sacrifice. After sacrifice, a circular ear punch biopsy (4 mm²) was taken, weighed, and fixed in formalin. Histological evaluation included H&E staining and immunohistochemical staining for CD45 to quantify the number of infiltrating immune cells (see below).

CD45 Immunohistochemical Staining—

Sections (10 μm) were cut from formalin-fixed paraffin-embedded ear biopsies and deparaffinized and rehydrated as described in (28). After antigen retrieval, inhibition of endogenous peroxidase with hydrogen peroxide, and blocking of non-specific antibody binding, sections were incubated with anti-CD45 antibody (BD Pharmingen, Franklin Lakes, N.J.) and visualized with diaminobenzidine as per the manufacturer's instructions using the ABC staining kit (Santa Cruz Biotech, Santa Cruz, Calif.). Some of the staining was performed by Georgia Pathology Research Services (Augusta, Ga.) using standard protocols. Multiple random sections (4 to 8 per mouse separately from the left and right ear) were counted by two independent observers in a blinded fashion, counts averaged to determine a value for each mouse ear and values statistically analyzed as described below.

Quantitative RT-PCR (qRT-PCR)—

Keratinocytes were treated with or without TPA or a synthetic triacylated lipoprotein (Pam₃CSK₄), a TLR1/TLR2 agonist, or recombinant S100A9 (R&D Systems) or HSPB4 (MyBiosoruce) as indicated, for 2 hours in the presence and absence of different concentrations of soy PG or dioleoylphosphatidylglycerol (DOPG). Cells were harvested and RNA isolated using PerfectPure RNA tissue kits (5 PRIME, Inc, Gaithersburg, Md., USA) as per the manufacturer's protocol. Following verification of RNA integrity using a Nanodrop instrument (NanoDrop Technologies, Wilmington, Del.), RNA was reverse-transcribed to cDNA with iScript cDNA synthesis kits (Bio-Rad Laboratories, Hercules, Calif., USA) and analyzed by real-time PCR using Taqman primer/probe sets from Applied Biosystems (Life Technologies, Grand Island, N.Y.). Reactions were performed using Fast Reagent PCR Master Mix (Applied Biosystems) and a StepOnePlus Real-Time PCR System (Applied Biosystems) as instructed by the manufacturer. Expression of genes of interest was determined using the delta-delta Ct method normalized to the expression of GAPDH as the endogenous housekeeping gene and expressed relative to the appropriate control group.

ELISA—

Cells were treated with recombinant S100A9 or recombinant HSPB4 as indicated and media collected. TNFα levels were determined in the supernatant using an ELISA kit (BD Biosciences) according to the manufacturer's instructions.

Western analysis—After completion of treatments, cells were harvested using hot lysis buffer [0.1875M Tris-HCl (pH 8.5), 3% SDS, and 1.5 mM EDTA]. After 5 minutes of incubation at room temperature, the solubilized cells were homogenized by repeated pipetting, and were mixed with 3× sample buffer (30% glycerol, 15% beta-mercaptoethanol, 1% bromophenol blue, 54% water) and boiled. For SDS-PAGE, equal volumes of the sample proteins were loaded on to 4-15% precast gels (Bio-Rad) and then transferred to PVDF membrane using a Trans-Blot Turbo Transfer system (Bio-Rad). Membranes containing transferred proteins were blocked with 5% non-fat dry milk for at least 30 minutes before incubating with the appropriate primary antibodies, overnight, followed by washing and incubating with secondary antibodies for 1 h at room temperature. Immunoreactivity was visualized using infrared imaging on an Odyssey imaging system (LI-COR Biosciences, Lincoln, Nebr.).

Statistical Analyses—

Statistical analyses were performed using ANOVA followed by Student-Newman-Keuls post-hoc tests, as performed by GraphPad Prism and GraphPad Instat (La Jolla, Calif.), with significance established at p≦0.05.

Results

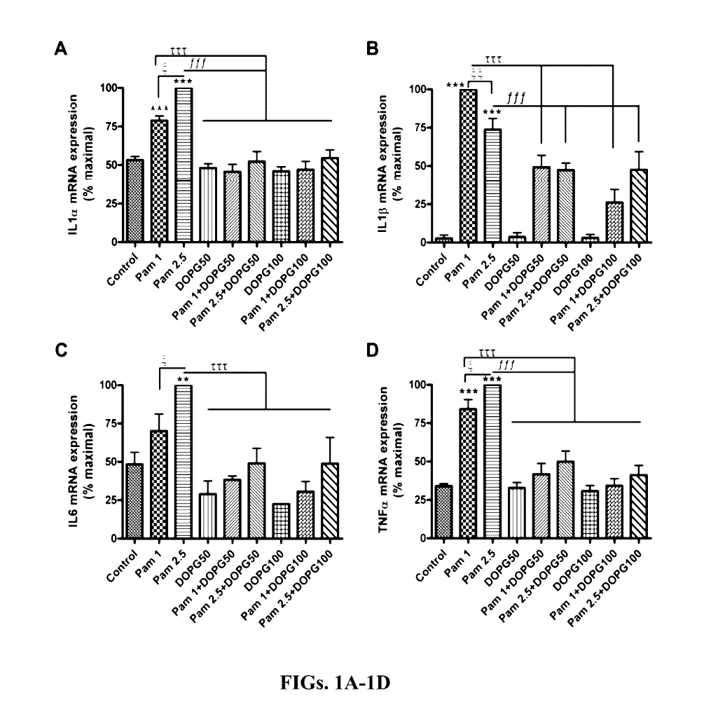

The ability of the phosphatidylglycerol (PG), dioleoylphosphatidylglycerol (DOPG, which has two 18-carbon fatty acids with one double bond each) to inhibit the expression of inflammatory mediators induced by an agonist of toll-like receptor-2 (TLR2) was examined. TLR1 is a pattern recognition receptor that is activated by microbial products and is involved in the innate immune response. A TLR1/2 agonist Pam₃CSK₄ was used, alone and in combination with DOPG, and expression levels of inflammatory mediators in keratinocytes were monitored. As seen in FIGS. 1A-1D, DOPG at either 50 or 100 μg/mL significantly inhibited the Pam3CSK4-induced increase in the expression of all cytokines tested.

### Example 2: Soy PG Inhibited Keratinocyte Inflammatory Mediator Expression in Response to Pam₃CSK₄

### Results

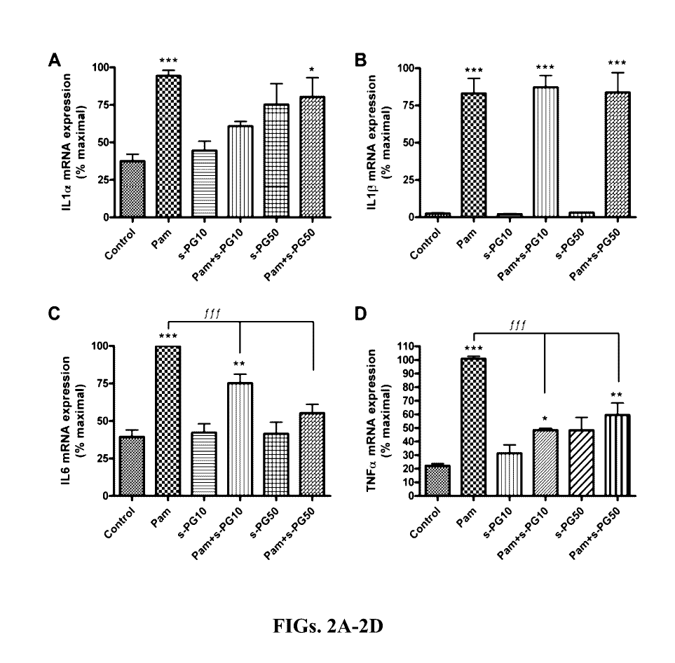

Similar effects on TLR1/2 agonist-induced IL-6 and TNFα expression were observed using soy PG (FIGS. 2A-2D), but soy PG alone seemed to have no effect on IL-1. Lower concentrations of soy PG were used based on our previous finding that soy PG was extremely potent at inhibiting keratinocyte proliferation (Xie D et al., PloS one, 9, e107119 (2014)). However, the ability to stimulate the expression of IL-1α at low concentrations, as well as other cytokines at higher doses, suggests that this particular PG species may not be optimal as an anti-inflammatory at concentrations tested.

### Example 3: DOPG Inhibits Pam₃CSK4-Induced Inflammatory Mediator Expression in the Macrophage Cell Line RAW 264.7

### Results

Although keratinocytes and keratinocyte-produced inflammatory mediators likely play an important role in inflammatory skin diseases, many such diseases, including psoriasis, are considered immune-mediated disorders (Lowes M A et al., Trends in immunology, 34, 174-181(2013); Sabat R et al., Journal of the German Society of Dermatology: JDDG, 9, 518-523 (2011); Brotas A M et al., Anais brasileiros de dermatologia 87, 673-681; quiz 682-673 (2012)). In addition, sterile sustained inflammation in the corneal epithelium was shown to be mediated by macrophage-released inflammatory mediators (Oh J Y et al., EMBO molecular medicine 4, 435-448 (2012)). Therefore, the action of PG on inflammatory mediator expression in an immune cell and selected the macrophage cell line, RAW 264.7 (RAW) was studied.

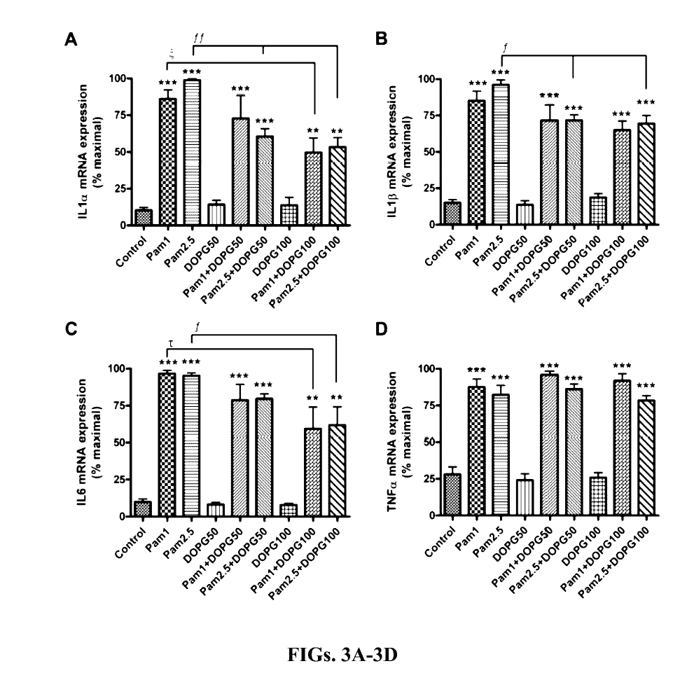

Similarly to the keratinocytes, upon stimulation with Pam₃CSK₄, RAW cells up-regulated their mRNA levels of IL1α, IL1β, IL6 and TNFα (FIGS. 3A-3D). Co-treatment with DOPG (100 μg/mL) resulted in an inhibition of IL1α, IL1β and IL6 mRNA levels which were induced by Pam₃CSK₄; the lower (50 μg/mL) DOPG concentration also inhibited IL1α and IL1β expression. However, DOPG had no effect on the Pam3CSK4 induction of TNFα, although this may be related to the relatively high concentrations of TLR1/2 agonist used. These results suggest that PG might be able to suppress inflammation by inhibiting TLR2 activation not only in keratinocytes but also in macrophages.

### Example 4: Recombinant S100A9 Induces the Expression of Inflammatory Mediators in Keratinocytes

### Results

Although activation of the immune system by the binding of microbial products is desired in the case of pathogens, not all microorganisms are pathogenic and thereby requiring immune system action. One way that the innate immune system can distinguish between dangerous and benign microbes is through their ability to injure cells. Cell injury results in the release of endogenous proteins, some of which, the so-called damage-associate molecular patterns or DAMPs, are now known to activate the innate immune system through toll-like receptors such as TLR2.

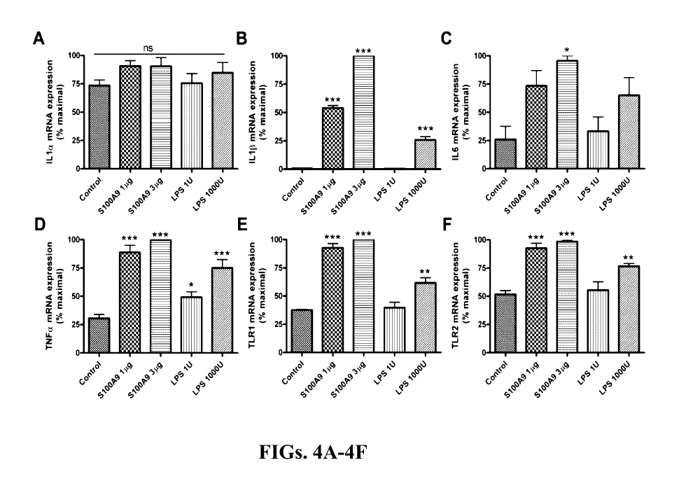

S100A9 is an anti-microbial protein produced at high levels in psoriasis and psoriasis models (Nakajima K et al., Journal of immunology, 186, 4481-4489 (2011); Zenz R et al., Nature, 437, 369-375 (2005); Gudjonsson J E et al., European journal of human genetics: EJHG, 14, 2-4 (2006); Schonthaler H B et al., Immunity, 39, 1171-1181 (2013)) and is known to be a DAMP that activates TLR2 (as well as TLR4) (Chen B et al., PloS one, 10, e0115828 (2015); Kang J H et al., Immunology, 144, 79-90 (2015); Schelbergen R F et al., Arthritis and rheumatism, 64, 1477-1487 (2012); Moles A et al., Journal of hepatology, 60, 782-791 (2014); Schiopu A et al., Mediators of inflammation, 2013, 828354 (2013)). The ability of recombinant S100A9 to trigger the expression of inflammatory mediators in keratinocytes was examined. In initial experiments, recombinant S100A9 protein at relatively low concentrations (1 to 3 μg/mL) stimulated the expression of IL1β, IL6 and TNFα (FIGS. 4B-4D).

The amount of endotoxin present in the recombinant S100A9 preparation, according to the supplier, is less than 0.1 U/μg protein. Lipopolysaccharide (LPS) was used at a comparable dose to determine if the contaminating endotoxin could explain the induction of inflammatory mediators by recombinant S100A9. However, at the doses of S100A9 used, any contaminating endotoxin would be expected to have little effect, based on the fact that concentrations of LPS of approximately 3 to 10 times the contaminating amount (i.e., 1 U/mL LPS) had a little or no effect, depending on the inflammatory mediator examined. Indeed, a dose approximately 1000 times the contaminating amount was required to observe a comparable stimulation of cytokine expression (FIGS. 4E-4F). In addition, denatured (heated) S100A9 was significantly less effective at stimulating inflammatory mediator mRNA levels, suggesting again that the protein itself, and not a contaminating microbial product, is responsible for recombinant S100A9's effect.

### Example 5: DOPG Inhibits Keratinocyte Expression of Inflammatory Mediators Induced by Recombinant S100A9

### Results

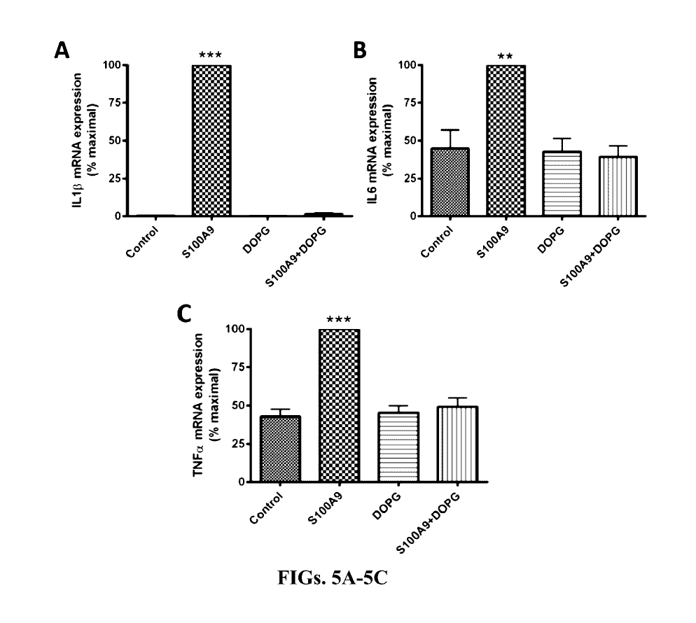

The ability of PG to inhibit S100A9-induced inflammatory mediator expression in keratinocytes was investigated. As shown in FIGS. 5A-5C, PG inhibited the expression of IL-1β, IL-6 and TNFα in keratinocytes stimulated with recombinant S100A9. As seen in the previous experiments, IL-1α was not investigated further since S100A9 only minimally stimulated IL-1α expression. Importantly, these results suggest the possibility of using PG as an anti-inflammatory therapy to treat psoriasis. It should also be noted that recombinant S100A9 induced the expression of TLR1 and TLR2 (data not shown), suggesting that this protein may potentially initiate a feed-forward effect that could enhance TLR1/2 signaling and exacerbate the production of inflammatory mediators.

### Example 6: DOPG Inhibits Expression of Inflammatory Mediators Induced by Recombinant S100A9 or Recombinant HSPB4 in a Macrophage Cell Line

### Methods and Materials

TNFα Protein Expression by ELISA

Mouse TNFα ELISA kits from BD Biosciences (San Jose, Calif.) were used. RAW264.7 cells were treated with or without recombinant S100A9 or HSPB4 in the presence and absence of DOPG for 2 hours. Cell culture supernatants were collected and stored at −80° C. and subjected to a single freeze-thaw cycle. The ELISA assay was performed according to the manufacturer's instructions with the optical density detected in each sample within 30 minutes, at a wavelength of 450 nm and corrected at 570 nm.

### Results

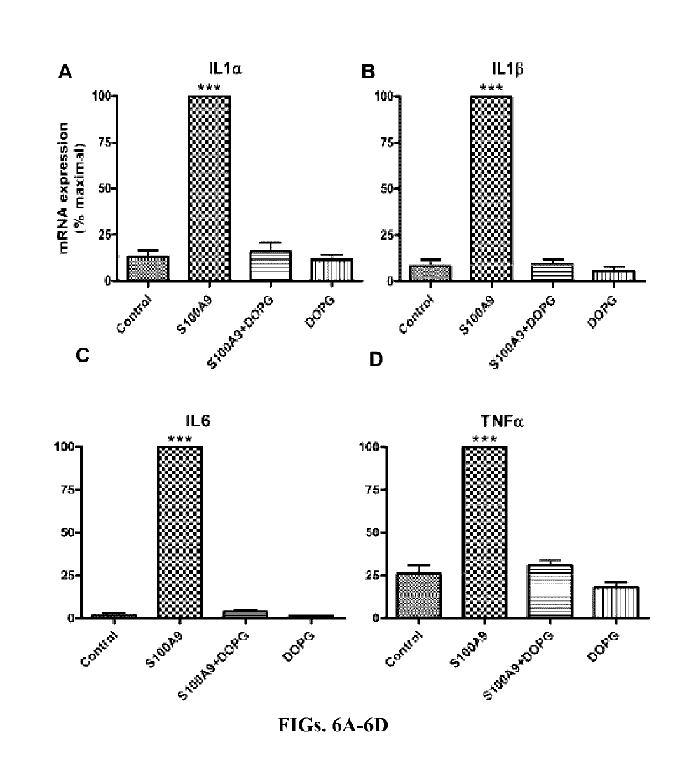

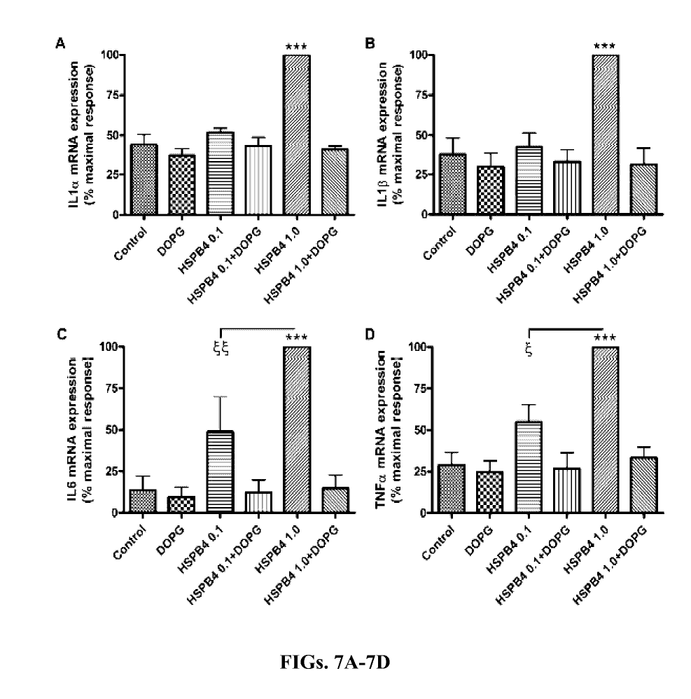

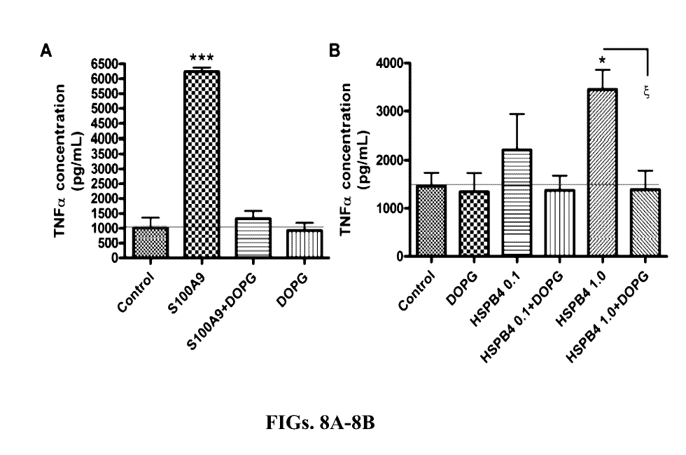

Data showed that S100A9 increases inflammatory mediator expression in the RAW 264.7 macrophage cell line and that DOPG can inhibit this increase (FIGS. 6A-6D). These data are consistent with the report that TLR2 activation by DAMPs depends upon PG-binding CD14 and that CD14 is necessary for NF-κB activation in response to DAMPs (Chun K H et al., International immunopharmacology, 10, 98-106 (2010); Kuronuma K et al., The Journal of biological chemistry 284, 25488-25500 (2009)). Furthermore, it was also found that HSPB4, another DAMP shown to mediate sterile corneal inflammation (Oh J Y et al., EMBO molecular medicine 4, 435-448 (2012)), could stimulate inflammatory mediator expression in the RAW cell line and, more importantly, that DOPG inhibited this induction (FIGS. 7A-7D). In addition, it was shown that the increases in mRNA expression are translated into enhanced cytokine secretion, at least for TNFα (the only one tested to date). Thus, both S100A9 and HSPB4 induce TNFα protein secretion and this increase is inhibited by DOPG (FIGS. 8A-8B).

These results suggest that DOPG can inhibit DAMP-induced inflammation and support the possibility of using DOPG as a therapy to treat sterile corneal inflammation mediated by TLR2 activation by HSPB4 in macrophages or to inhibit inflammation induced by anti-microbial proteins such as S100A9 in psoriasis.

### Example 7: DOPG Inhibits Pam-Induced NF-kB Phosphorylation/Activation

### Results

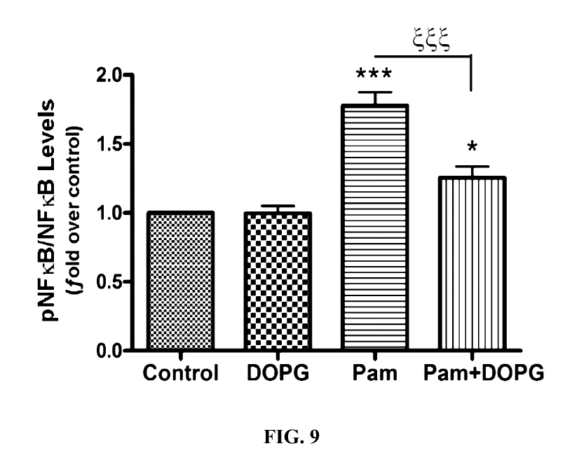

The effect of DOPG on the activation of nuclear factor-kappa B (NF-κB) in RAW264.7 cells stimulated with Pam₃CSK₄ was also investigated. NF-κB is a transcription factor downstream of toll-like receptors and known for its pro-inflammatory actions. As shown in FIG. 9, Pam₃CSK₄ increased the phosphorylation/activation of NF-κB whereas co-treatment with DOPG inhibited this effect. Again, this result is consistent with an anti-inflammatory action of PG.

### Example 8: PG Inhibits Inflammation in a Contact Irritant Ear Edema Inflammation Model In Vivo

### Methods and Materials

Contact Irritant Ear Edema Mouse In Vivo Model

Experiments were performed as described in Clark S P; Bollag W B; Westlund K N; Ma F; Falls G; Xie D; Johnson M; Isales C M; Bhattacharyya M H: Pine oil effects on chemical and thermal injury in mice and cultured mouse dorsal root ganglion neurons. Phytother Res 2014, 28, 252-269. Briefly, the ears of three to five male ICR CD-1 outbred mice [25-30 g; 5-6 weeks of age from Harlan Laboratories (Indianapolis, Ind.)] were treated with acetone or TPA (0.03% in acetone), and at 1 h and 4h after TPA, the appropriate vehicle or the treatment in vehicle was applied to each ear. Ear thickness was measured using a caliper both before and at approximately 18-20 h after TPA treatment prior to sacrifice. After sacrifice, a circular ear punch biopsy (4 mm2) was taken, weighed, and fixed in formalin. Histological evaluation included H&E staining and immunohistochemical staining for CD45 to quantify the number of infiltrating immune cells (see below).

CD45 Immunohistochemical Staining

Sections (10 μm) were cut from formalin-fixed paraffin-embedded ear biopsies and deparaffinized and rehydrated as described in (Voss K E et al., Arch. Dermatol. Res. 303 591-600 (2011)). After antigen retrieval, inhibition of endogenous peroxidase with hydrogen peroxide, and blocking of non-specific antibody binding, sections were incubated with anti-CD45 antibody (BD Pharmingen, Franklin Lakes, N.J.) and visualized with diaminobenzidine as per the manufacturer's instructions using the ABC staining kit (Santa Cruz Biotech, Santa Cruz, Calif.). Some of the staining was performed by Georgia Pathology Research Services (Augusta, Ga.) using standard protocols. Multiple random sections (4 to 8 per mouse separately from the left and right ear) were counted by two independent observers in a blinded fashion, counts averaged to determine a value for each mouse ear and values statistically analyzed as described below.

Mice Treated with PG and/or 1,25-Dihydroxyvitamin D3

Ear thickness was measured with a digital caliper prior to treatment of both ears with 0.03% TPA in acetone (or acetone alone). One and four hours after the TPA application, vehicle (95% ethanol/5% water) or vehicle containing PG or 1,25-dihydroxyvitamin D3 (VitD) or the combination was applied to the ears and 20 hours later ear thickness was measured again. The change in ear thickness was calculated as the thickness at the end of the experiment minus the thickness of the same ear measured at time zero.

After TPA and treatment applications as described above, mice were sacrificed and a 4 mm punch biopsy was harvested from each ear and weighed. The change in ear weight was calculated as the weight measured at sacrifice minus the average weight of the ear biopsies from the untreated controls.

Multiple sections were cut from formalin-fixed, paraffin-embedded ear biopsies and stained for CD45 as described in Materials and Methods. The number of CD45-positive cells was determined by counting immunostained cells in at least five random fields from a minimum of two sections from the left and right ear biopsies of treated mice. Cells were counted in a blinded manner by at least two independent observers and the counts were averaged for each mouse.

Mice Treated with PG in Penetration-Enhancing Vehicle

Ear thickness and weight were measured with a digital caliper prior to application of TPA in acetone (time zero) to both surfaces (inner and outer) of the ear. Vehicle (octanoic acid with added stearic acid to yield the desired consistency) with or without the indicated amounts of soy PG was applied to the ears 1 and 4 hours after TPA treatment. Ear thickness was determined again approximately 18 hours after the initial exposure to TPA, and the percent change from time 0 was calculated. For the weight measurements punch biopsies were taken from each ear and weighed. Some mice received no TPA and the average weight of the ear punch biopsies of these mice was used for calculations as the time 0 value.

### Results

To determine if PG is effective in vivo against inflammation, the effect of soy PG was tested on the contact irritant mouse ear edema model, used to mimic the inflammation associated with psoriasis. In this case, soy PG, rather than DOPG was selected, because it would have the benefit in psoriasis not only of reducing inflammation but also of inhibiting the keratinocyte hyperproliferation that characterizes this skin disease. Thus, 12-O-tetra-decanoylphorbol 13-acetate (TPA) was applied to the ears of mice, and treated with vehicle, soy PG, 1,25-dihydroxyvitamin D3, or the combination, and the edema was monitored as changes in weight or in ear thickness (using calipers).

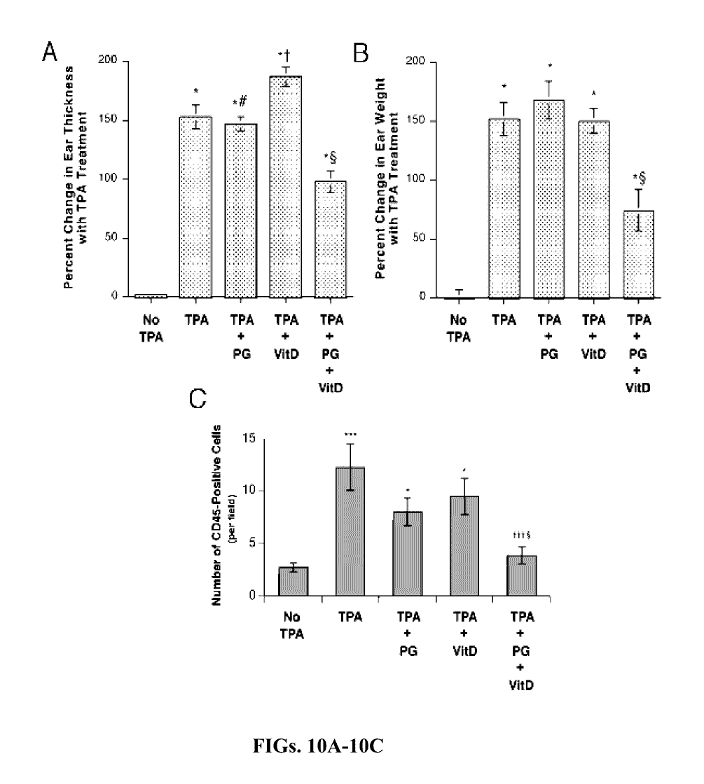

As shown in FIGS. 10A-10C, neither 1,25-dihydroxyvitamin D3 nor soy PG alone had a significant effect on ear edema. However, the combination significantly inhibited both the TPA-induced increase in ear thickness and weight (FIGS. 10A and 10B), as well as the immune cell infiltration induced by the contact irritant (as measured by the infiltration of CD45-positive cells, FIG. 10C). The results also suggest that the combination of 1,25-dihydroxyvitamin D3 and soy PG inhibits the TPA-induced levels of tumor necrosis factor-α (data not shown).

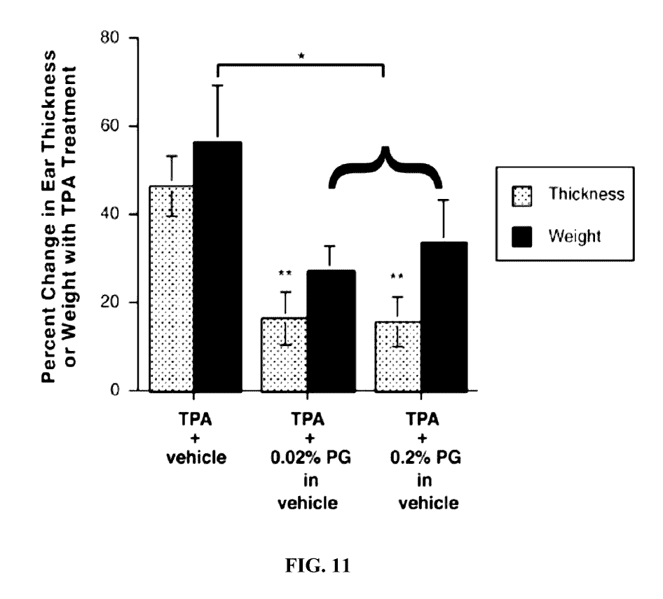

Since acute exposure to 1,25-dihydroxyvitamin D3 is known to disrupt the epidermal barrier (von Brenken S et al., Dermatology 194, 151-156(1997)), this result suggested the possibility that soy PG itself was not effective in inhibiting ear edema because it was unable to penetrate the barrier. Therefore, the experiment was repeated by applying soy PG (two doses of 0.02 and 0.2%) in a penetration-enhancing vehicle (a cream of trioctanoin magnesium-stearate). In this case, although it was found that the vehicle itself had a significant inhibitory effect on TPA-induced changes in ear thickness and weight, soy PG significantly inhibited ear edema over and above the inhibition induced by vehicle (FIG. 11). TPA alone (with no subsequent treatment with vehicle) induced an increase in ear thickness of 147±16% (versus a 1.7±2% change with no TPA treatment) and in weight of 158±6% (p<0.05 versus no treatment and versus TPA+vehicle, which corresponds well with the value obtained in experiment shown in FIGS. 10A-10B.

It should be noted that excipients (vehicles) have previously been shown to have an effect on skin function (reviewed in Surber C et al., Dermatology, 210, 157-168 (2005)); nevertheless, vehicle containing soy PG was more efficacious than vehicle alone, indicating that soy PG is able to inhibit inflammation in the epidermis. Together these results indicate an anti-inflammatory action of PG in vivo.

The results indicate that soy PG, applied either together with 1,25-dihydroxyvitamin D3 or alone in a penetration-enhancing vehicle, suppresses inflammation induced by the phorbol ester. Topical TPA is known to increase epidermal S100A proteins. S100A proteins are consistently up-regulated in psoriasis and can activate toll-like receptor-2 (TLR2), a member of the innate immune system which can elicit inflammation. Indeed, keratinocytes treated in vitro with a TLR2 agonist (Pam₃CSK₄) exhibited increased inflammatory mediator expression, and treatment of keratinocytes with PG inhibited this expression. The experiments showed results that are consistent with those using recombinant S100A9 protein. The data thus support using PG as a topical treatment for psoriasis.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method for treating psoriasis in a subject in need thereof comprising:
administering to the subject an effective amount of phosphatidylglycerol to reduce or inhibit inflammation associated with the psoriasis.

2. The method of claim 1, wherein the phosphatidylglycerol is topically administered to a site of psoriasis on the subject.

3. The method of claim 1, wherein the subject is human.

4. The method of claim 1, wherein the phosphatidylglycerol is soy phosphatidylglycerol.

5. The method of claim 1, wherein the effective amount of phosphatidylglycerol consists essentially of phosphatidylglycerol.

6. The method of claim 1, wherein the effective amount of phosphatidylglycerol is in an amount effective to inhibit or reduce inflammatory mediator expression induced by S100A9 protein.

7. The method of claim 1, wherein the effective amount of phosphatidylglycerol is in an amount effective to inhibit or reduce toll-like receptor 2 activation.

8. A method of treating inflammation in the eye of subject comprising:
administering to the eye of the subject an effective amount of phosphatidylglycerol to reduce or inhibit toll-like receptor 2 activation in the eye of the subject.

9. The method of claim 8, wherein the phosphatidylglycerol is soy phosphatidylglycerol.

10. The method of claim 8, wherein the effective amount of phosphatidylglycerol consists essentially of phosphatidylglycerol.

11. A pharmaceutical composition for treating inflammation in a subject, comprising:
an active agent comprising phosphatidylglycerol in an amount effective to inhibit or reduce toll-like receptor 2 activation in the subject and thereby inhibit or reduce inflammation in the subject.

12. A pharmaceutical composition for treating inflammation in a subject, comprising:
an active agent consisting essentially of phosphatidylglycerol to inhibit or reduce toll-like receptor 2 activation in the subject and thereby or reduce inflammation in the subject.

13. The pharmaceutical composition of claim 11 or 12, wherein the composition further comprises a barrier-disrupting agent or a penetration-enhancing vehicle.

14. The pharmaceutical composition of claim 13, wherein the barrier-disrupting agent or penetration-enhancing vehicle is selected from the group consisting of a humectant, powder, oil/water (O/W) emulsion, water/oil emulsion, absorption base, fats, waxes, oils, silicones, salicylic acid, urea phospholipase A2, phosphatidylcholine dependent phospholipase C, ethanol, acetone, detergents, bases, propylene glycol, pyrriolidones, dimethylacetamide, dimethylformamide, dimethylsulfoxide, alkyl sulfoxide, phosphine oxide, surfactants and caprolactams such as azone, amines and amides, alkyl N,N-distributed-amino acetates, decylmethylsulfoxide, pyrrolidones, pirotiodecane (HPE-101), benzlyalkonium, benzylalkonium chloride polymers, silicone based polymers, fatty acids, cyclic ureas, terpenes, liposomes, cyclodextrins, and combinations thereof.
